(19) United States
(12) Patent Application Publication
(10) Pub. No.: US 2024/0005489 A1
(43) Pub. Date: Jan. 4, 2024

(54) MEASUREMENT DEVICE, MEASUREMENT METHOD, AND STORAGE MEDIUM

(71) Applicant: SHARP KABUSHIKI KAISHA, Sakai City (JP)

(72) Inventors: Kyohko Matsuda, Sakai City (JP); Yoshihisa Adachi, Sakai City (JP); Ryota Tomizawa, Sakai City (JP)

(21) Appl. No.: 18/210,599

(22) Filed: Jun. 15, 2023

(30) Foreign Application Priority Data
Jun. 29, 2022 (JP) 2022-104103

(51) Int. Cl. G06T 7/00; A61B 5/00

(52) U.S. Cl. CPC G06T 7/0012; A61B 5/0077; A61B 5/7485; G06T 2207/30004; G06T 2207/30196; G06T 2207/30168; G06T 2207/10024

(57) ABSTRACT

A measurement device includes the following: an imaging unit configured to image a living organism to acquire an image; a determination-index calculating unit configured to calculate, for a region of interest included in the image, a determination index relating to a pixel count of a target pixel having a pixel value equal to or greater than a predetermined lower limit and equal to or smaller than a predetermined upper limit; and a biological-information calculating unit configured to calculate biological information from the pixel value of a pixel included in a region of interest with the determination index being equal to or greater than a threshold.

### BACKGROUND

### Field of the Disclosure

The present disclosure relates to a measurement device, a measurement method, and a program. The present application claims priority from Japanese Application No. JP2022-104103, filed on Jun. 29, 2022, the content of which is hereby incorporated by reference into this application.

### Description of the Related Art

Japanese Unexamined Patent Application Publication No. 2016-190022 discloses a technique of calculating the difference between luminance information about a video signal in a skin region indicating the face of a target person and luminance information about a video signal in a skin region indicating parts of the target person other than the face at the same time as the video signal of the face, and measuring fluctuations in the blood pressure of the target person corresponding to increase or decrease in the calculated difference.

### SUMMARY

One method is calculating biological information by imaging skin to acquire information about the skin surface, the skin inner blood vessels and other things. For instance, the technique disclosed in Japanese Unexamined Patent Application Publication No. 2016-190022 includes extracting pixels of luminance information corresponding to skin color from a region of interest set manually, to set a region including the extracted pixels as a skin region. However, the biological information cannot be calculated accurately in some cases in the technique disclosed in Japanese Unexamined Patent Application Publication No. 2016-190022 when the skin region includes inappropriate pixels containing information irrelevant to the blood vessels, such as a blocked-up shadow image, caused by excessively dark surroundings, a spot image, a wrinkle image and other things. The reason is that such an inappropriate pixel containing information unnecessary for the calculation of biological information calculation cannot reflect the biological information correctly; thus, it is difficult to calculate the biological information accurately when an image includes many inappropriate pixels like this. Examples of the inappropriate pixel include a pixel with a blocked-up shadow, caused by excessively dark surroundings as described above, a pixel darkened due to, but not limited to, a spot, a wrinkle, a moles or a face shadow, and a pixel with an image showing blown-out highlights due to excessively intensive illumination or sunlight.

Accordingly, one aspect of the present disclosure aims to provide a measurement device, a measurement method, and a recording medium that can calculate biological information accurately by using an appropriate pixel.

A measurement device according to one aspect of the present disclosure includes the following: an imaging unit configured to image a living organism to acquire an image; a determination-index calculating unit configured to calculate, for a region of interest included in the image, a determination index relating to a pixel count of a target pixel having a pixel value equal to or greater than a predetermined lower limit and equal to or smaller than a predetermined upper limit; and a biological-information calculating unit configured to calculate biological information from the pixel value of a pixel included in a region of interest with the determination index being equal to or greater than a threshold.

A measurement method according to one aspect of the present disclosure includes the following steps: imaging a living organism to acquire an image; calculating, for a region of interest included in the image, a determination index relating to a pixel count of a target pixel having a pixel value equal to or greater than a predetermined lower limit and equal to or smaller than a predetermined upper limit; and calculating biological information from the pixel value of a pixel included in a region of interest with the determination index being equal to or greater than a threshold.

A computer-readable recording medium according to one aspect of the present disclosure records a program that is executed by a computer. The program includes the following functions: imaging a living organism to acquire an image; calculating, for a region of interest included in the image, a determination index relating to a pixel count of a target pixel having a pixel value equal to or greater than a predetermined lower limit and equal to or smaller than a predetermined upper limit; and calculating biological information from the pixel value of a pixel included in a region of interest with the determination index being equal to or greater than a threshold.

### BRIEF DESCRIPTION OF THE DRAWINGS

FIG. 1 illustrates an example aspect of use of a measurement device;

FIG. 2 is a block diagram illustrating an example configuration of the measurement device;

FIG. 3 is a flowchart illustrating an example operation of the measurement device according to a first preferred embodiment;

FIG. 4 is a flowchart illustrating an example operation of the measurement device according to the first preferred embodiment subsequent to FIG. 3;

FIG. 5 illustrates example target pixels and example non-target pixels included in a region of interest;

FIG. 6 illustrates a threshold and a graph showing example time changes in a determination index;

FIG. 7 is a flowchart illustrating an example operation of a measurement device according to a second preferred embodiment;

FIG. 8 is a flowchart illustrating an example operation of the measurement device according to the second preferred embodiment subsequent to FIG. 7;

FIG. 9 illustrates a threshold, a total-use threshold, and a graph showing example time changes in a determination index;

FIG. 10 illustrates a plurality of example regions of interest included in an image; and

FIG. 11 illustrates a plurality of example regions of interest included in a region of interest.

### DETAILED DESCRIPTION OF THE DISCLOSURE

### First Preferred Embodiment

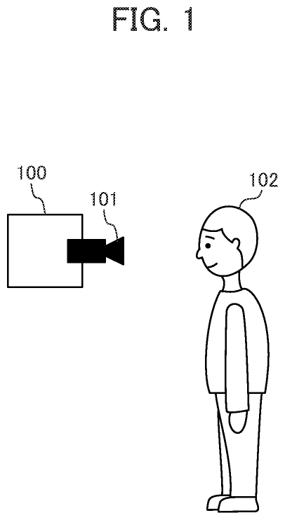

A first preferred embodiment will be described with reference to FIG. 1 to FIG. 6. It is noted that identical or equivalent components in the drawings will be denoted by the same signs, and that the description of redundancies will be omitted.

FIG. 1 illustrates an example aspect of use of a measurement device 100. As illustrated in FIG. 1, the measurement device 100 includes an imaging unit 101.

The measurement device 100 measures time-series changes in the skin's surface and inside of a living organism 102 from an image acquired by the imaging unit 101, to acquire biological information. Examples of the measurement device 100 include a personal computer (PC), a smartphone, a tablet terminal, a terminal dedicated for biological-information measurement, and a monitoring robot provided with the imaging unit 101. Examples of the biological information include blood pressure, pulse, breathing rate, and blood oxygen saturation.

The imaging unit 101 images the living organism 102 to acquire an image. In the present disclosure, still and moving images cut out of a continuous or discontinuous live-action record indicating volume changes in a blood vessel of the living organism 102 imaged by the imaging unit 101 are referred to as images.

The imaging unit 101 is placed in a location where the imaging unit 101 can image a portion where the body surface of the living organism 102 is exposed. Examples of the portion where the body surface of the living organism 102 is exposed include the forehead, cheek, fingertips, wrists and palms of the living organism 102. The imaging unit 101 is placed in, but not limited to, a PC, a display, a mirror, and a washstand.

The imaging unit 101 is a camera that includes a charged-coupled-device (CCD) image sensor or complementary-metal-oxide-semiconductor (CMOS) image sensor, and a lens. The imaging unit 101 may be composed of a camera image sensor including an RGB filter. The imaging unit 101 includes, for instance, a color filter of RGB Bayer arrangement in order to detect a small change in the skin color of the living organism 102. Alternatively, the imaging unit 101 may include a color filter of RGBCy, RGBIR or other things. The color filter of RGBCy, RGBIR or other things is suitable for observing increase and decrease in blood volume indicated by reflected light of light passed through the skin's inside.

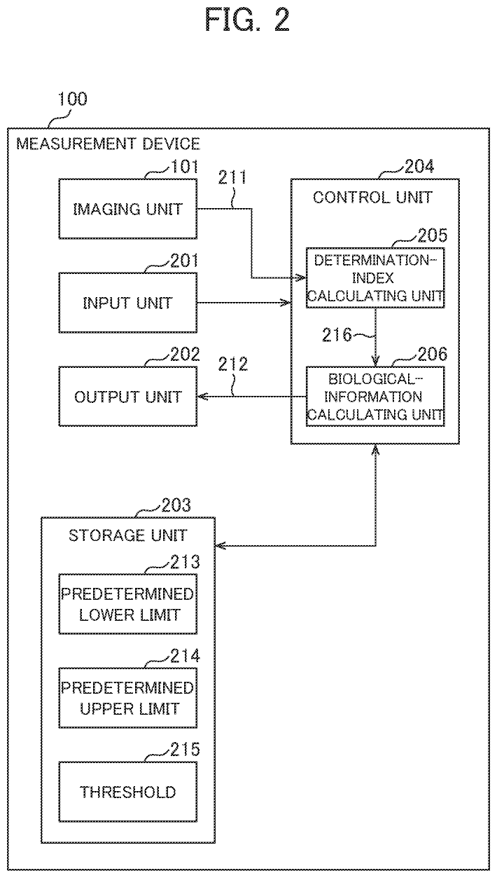

FIG. 2 is a block diagram illustrating an example configuration of the measurement device 100. The measurement device 100 includes the imaging unit 101, an input unit 201, an output unit 202, a storage unit 203, a control unit 204, a determination-index calculating unit 205, a biological-information calculating unit 206, and other components. The imaging unit 101, the input unit 201, the output unit 202 and the storage unit 203 are electrically connected to the control unit 204.

The imaging unit 101 images the living organism 102 to acquire an image 211 and sends the acquired image 211 to the control unit 204. The imaging unit 101 images the living organism 102 at, for instance, 30 to 60 frames per second (fps) to acquire the image 211. The image 211 includes an image of the body surface of the living organism 102.

The input unit 201 receives an input of information necessary for the measurement device 100. Examples of the input unit 201 include a keyboard, a mouse, and a touch panel.

The output unit 202 outputs the image 211, biological information 212, a message to the living organism 102, date and time, and other things. The output unit 202 is composed of, but not limited to, a display and a loudspeaker.

The storage unit 203 is a recording medium that can record various data pieces, various programs, and other things and is composed of, but not limited to, a hard disk, a solid-state drive (SSD), and a semiconductor memory. The storage unit 203 stores a predetermined lower limit 213, a predetermined upper limit 214, a threshold 215, information related to the biological information 212, time changes in the biological information 212, and other things. The information relating to the biological information 212 is the type of the biological information 212, logs of the biological information 212 (e.g., changes on a day-to-day basis), data for correcting information already stored in the storage unit 203, or other things.

The control unit 204 executes various processes in accordance with programs and data stored in the storage unit 203. The control unit 204 is composed of a processor, such as a central processing unit (CPU) or a graphic processing unit (GPU).

The control unit 204 includes the determination-index calculating unit 205, the biological-information calculating unit 206, and other components.

The determination-index calculating unit 205 calculates, for a region of interest included in the image 211, a determination index 216 relating to the number of target pixels TP having a pixel value equal to or greater than the predetermined lower limit 213 and equal to or smaller than the predetermined upper limit 214. A pixel value in the present disclosure is information indicating the brightness of a pixel included in the image 211; examples include the pixel value of a subpixel of each of red (R), green (G) and blue (B) in an RGB color space, the luminance value of a pixel in the RGB color space, and the values of hue (H), saturation (S) and value (V) calculated from the pixel values of the R-, G- and B-subpixels in an HSV color space.

The biological-information calculating unit 206 calculates the biological information 212 from the pixel value of a pixel included in a region of interest with the determination index 216 being equal to or greater than the threshold 215. To be specific, the biological-information calculating unit 206 calculates the biological information 212 from the pixel values of the target pixels TP included in the region of interest with the determination index 216 being equal to or greater than the threshold 215.

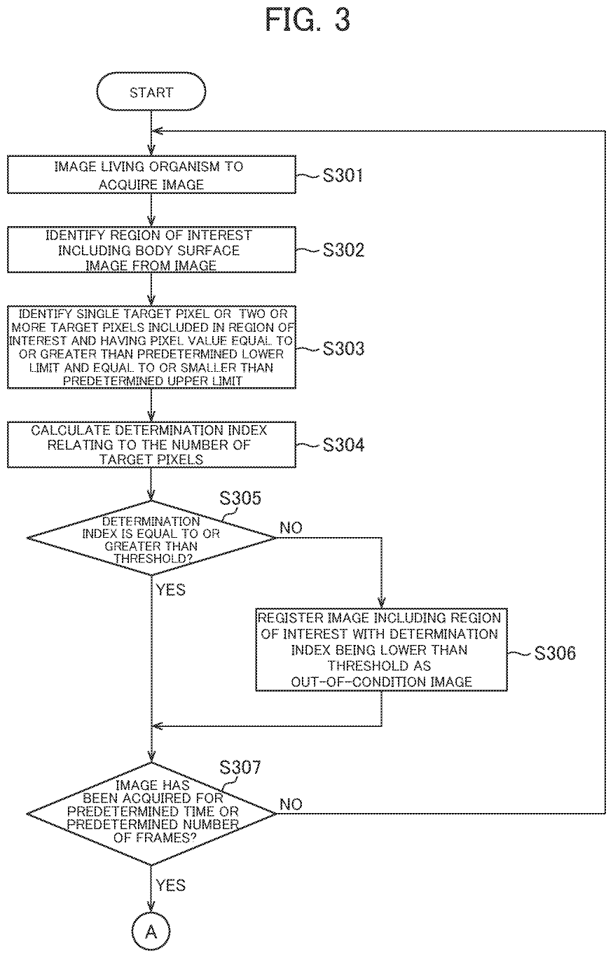

FIG. 3 is a flowchart illustrating an example operation of the measurement device 100 according to this preferred embodiment.

In Step S301, the imaging unit 101 images the living organism 102 to acquire the image 211. When the output unit 202 is implemented by a display, the control unit 204 controls the display to show the acquired image 211.

In Step S302, the determination-index calculating unit 205 identifies a region of interest including a body surface image from the acquired image 211. For instance, the determination-index calculating unit 205 detects a facial region of the living organism 102 from the acquired image 211. To be specific, the determination-index calculating unit 205 detects characteristics, such as the eyes, the nose and the mouth, from the image 211 and detects the facial region on the basis of the detected characteristics. The determination-index calculating unit 205 then identifies a region of interest including an image of the forehead or cheek of the living organism 102 from the detected facial region.

In Step S303, the determination-index calculating unit 205 identifies a single target pixel TP or two or more target pixels TP included in the identified region of interest and having a pixel value equal to or greater than the predetermined lower limit 213 and equal to or smaller than the predetermined upper limit 214.

For instance, the determination-index calculating unit 205 determines whether the pixel values of the R-, G- and B-subpixels constituting a pixel included in the region of interest is equal to or greater than the predetermined lower limit 213 and equal to or smaller than the predetermined upper limit 214. The determination-index calculating unit 205 then identifies, as a target pixel TP, a pixel with the pixel values of the R-, G- and B-subpixels being all equal to or greater than the predetermined lower limit 213 and equal to or smaller than the predetermined upper limit 214.

For instance, the predetermined lower limit 213 has a value corresponding to the lower limit of a dynamic range of the image 211, and the predetermined upper limit 214 has a value corresponding to the upper limit of the dynamic range of the image 211. To be specific, the predetermined lower limit 213 has a larger value than the lower limit of the dynamic range, and the predetermined upper limit 214 has a smaller value than the upper limit of the dynamic range.

For instance, let the predetermined lower limit 213 stand at one, and let the predetermined upper limit 214 stand at 254 when the lower limit, which is a pixel value corresponding to the lower limit of an RGB dynamic range, stands at zero, and when the upper limit, which is a pixel value corresponding to the upper limit of the RGB dynamic range, stands at 255. The determination-index calculating unit 205 in this case identifies, as target pixels TP, pixels included in the region of interest and having a pixel value of 1 to 254 inclusive. The determination-index calculating unit 205 can accordingly exclude a blocked-up shadow pixel having a pixel value of zero, and a blown-out highlight pixel having a pixel value of 255 from the target pixels TP. Alternatively, the predetermined lower limit 213 may have a larger value than the lower limit of the dynamic range by a predetermined value, and the predetermined upper limit 214 may be a smaller value than the upper limit of the dynamic range by a predetermined value. For instance, when the lower limit of the RGB dynamic range stands at zero, and when the upper limit of the RGB dynamic range stands at 255, the predetermined lower limit may stand at 10, and the predetermined upper limit may stand at 244. It is noted that the upper and lower limits of the dynamic range will be hereinafter indicated by corresponding pixel values, and that a grayscale, such as 12-bit, may be used other than a 256-level grayscale.

In Step S304, the determination-index calculating unit 205 calculates the determination index 216 relating to the number of target pixels TP identified in Step S303. For instance, the determination-index calculating unit 205 calculates the ratio of the target pixels TP to all the pixels in the region of interest as the determination index 216. For instance, let the region of interest include 100 pixels in total, and let 80 pixels have a pixel value equal to or greater than the predetermined lower limit 213 and equal to or smaller than the predetermined upper limit 214. The determination index 216 in this case stands at 80%. Alternatively, the determination-index calculating unit 205 may calculate the number of target pixels TP as the determination index 216. In Step S305, the biological-information calculating unit 206 determines whether the determination index 216 calculated in Step S304 is equal to or greater than the threshold 215. For instance, when the determination index 216 indicates the ratio of the target pixels TP to all the pixels in the region of interest, and when the threshold 215 stands at 60%, the biological-information calculating unit 206 determines whether the ratio of the target pixels TP indicated by the determination index 216 is equal to or greater than 60%.

If the determination index 216 is equal to or greater than the threshold 215 in Step S305, the control unit 204 moves the process to Step S307. In contrast, if the determination index 216 is lower than the threshold 215 in Step S305, the biological-information calculating unit 206 registers the image 211 including the region of interest with the determination index 216 being lower than the threshold 215 into the storage unit 203 as an out-of-condition image. For instance, the determination-index calculating unit 205 registers the identification information of the image 211 including the region of interest with the determination index 216 being lower than the threshold 215 into the storage unit 203 as the identification information of the out-of-condition image. An example of the identification information of the image 211 is a frame number. The control unit 204 then moves the process to Step S307.

In Step S307, the biological-information calculating unit 206 determines whether the image 211 has been acquired for a predetermined time or a predetermined number of frames. The predetermined time or the predetermined number of frames is the time or the number of frames necessary for calculating the biological information 212. The predetermined time or the predetermined number of frames may be different from type to type of the biological information 212.

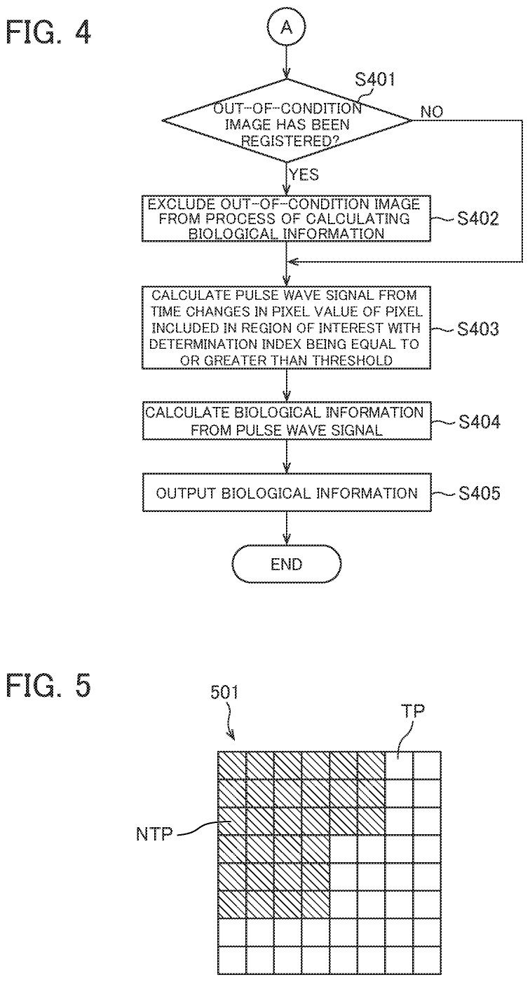

If the image 211 has not been acquired for the predetermined time or the predetermined number of frames in Step S307, the control unit 204 moves the process back to Step S301. In contrast, if the image 211 has been acquired for the predetermined time or the predetermined number of frames in Step S307, the control unit 204 moves the process to Step S401, which is illustrated in FIG. 4.

The operation of the measurement device 100 according to this preferred embodiment will be still described with reference to FIG. 4.

In Step S401, the biological-information calculating unit 206 determines whether an out-of-condition image has been registered. For instance, the biological-information calculating unit 206 determines whether the identification information of the out-of-condition image has been registered in the storage unit 203. If the identification information of the out-of-condition image has not been registered in the storage unit 203, the control unit 204 moves the process to Step S403. In contrast, if the identification information of the out-of-condition image has been registered in the storage unit 203, the biological-information calculating unit 206 in Step S402 excludes, among a plurality of images or images in a predetermined number of frames acquired by imaging the living organism 102 for a predetermined time, an out-of-condition image with identification information registered in the storage unit 203 being assigned thereto from the process of calculating the biological information 212. Alternatively, the biological-information calculating unit 206 may exclude a moving image including the out-of-condition image with the identification information registered in the storage unit 203 being assigned thereto from the process of calculating the biological information 212. That is, the biological-information calculating unit 206 determines an out-of-condition image including a region of interest with the determination index 216 being lower than the threshold 215, or a moving image including the out-of-condition image as an imaging failure and then excludes the out-of-condition image, or the moving image including the out-of-condition image from the process of calculating the biological information 212.

In Step S403, the biological-information calculating unit 206 calculates a pulse wave signal indicating a pulse wave from time changes in the pixel value of a pixel included in a region of interest with the determination index 216 being equal to or greater than the threshold 215. A pulse wave in the present disclosure is a time-series signal that indicates blood-vessel volumetric change, and that is calculated from a time-series signal indicating the pixel value of a pixel included in the image 211.

For instance, the biological-information calculating unit 206 calculates a representative value of pixel values of the pixels included in the region of interest with the determination index 216 being equal to or greater than the threshold 215. The biological-information calculating unit 206 then calculates the pulse wave signal from time changes in a value calculated by substituting the extracted pixel values, or the representative value of these pixel values into a predetermined mathematical expression. Alternatively, the biological-information calculating unit 206 may calculate, through independent component analysis, pigment composition separation or other methods, the pulse wave signal from time changes in the pixel value of the pixel included in the region of interest with the determination index 216 being equal to or greater than the threshold 215.

In Step S404, the biological-information calculating unit 206 calculates the biological information 212 corresponding to the type of the biological information 212, which is a calculation target, from the pulse wave signal calculated in Step S403. For instance, when the type of the calculation target, i.e., the biological information 212, is pulse rate, the biological-information calculating unit 206 calculates the pulse rate on the basis of the time required for a single beat of the pulse wave indicated by the pulse wave signal. Further for instance, when the type of the calculation target, i.e., the biological information 212, is blood pressure, the biological-information calculating unit 206 estimates the blood pressure from the shape of the pulse wave signal.

In Step S405, the biological-information calculating unit 206 controls the output unit 202 to output the biological information 212 calculated in Step S404.

For instance, when the output unit 202 is composed of a display, the biological-information calculating unit 206 controls the display to show characters, a graph or other things indicating the pulse rate, blood pressure or other things indicated by the biological information 212. Furthermore, when the output unit 202 is composed of a display, the biological-information calculating unit 106 may control the display to show a message to the living organism 102, date and time, and other things. An example message to the living organism 102 indicates whether the pulse rate, blood pressure or other things indicated by the biological information 212 falls within a predetermined range; a message saying, “a normal measurement” is an example of such a message. Alternatively, when the output unit 202 is composed of a loudspeaker for instance, sounds indicating, but not limited to, a message to the living organism 102, such as the pulse rate, blood pressure or other things indicated by the biological information 212, and date and time may be output from the loudspeaker.

FIG. 5 illustrates example target pixels TP and example non-target pixels NTP included in a region of interest 501. The non-target pixels NTP are pixels having a pixel value smaller than the predetermined lower limit 213 or greater than the predetermined upper limit 214.

The region of interest 501 includes 64 pixels in total, among which 34 pixels are the target pixels TP. Thus, the determination-index calculating unit 205 calculates the determination index 216 as standing at 53.125% (=34/64) when, for instance, calculating the ratio of the target pixels TP to all the pixels in the region of interest as the determination index 216.

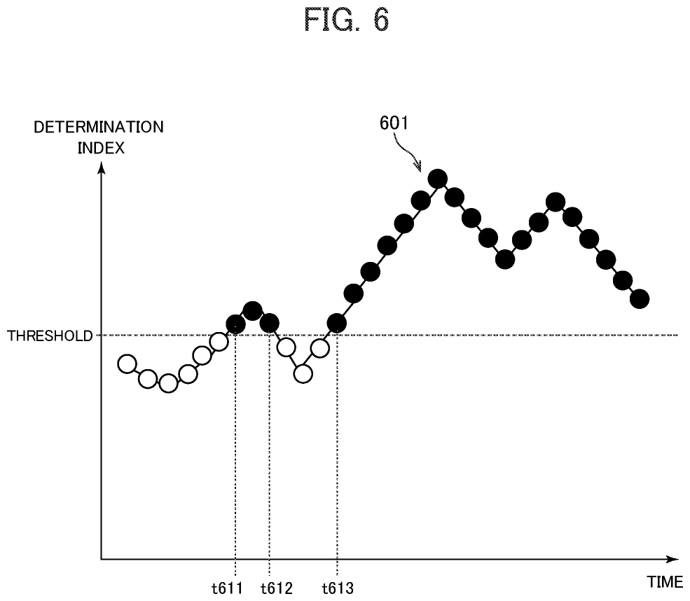

FIG. 6 illustrates the threshold 215, and a graph 601 showing example time changes in the determination index 216. The lateral axis in FIG. 6 indicates time, and the longitudinal axis in the same indicates the determination index. Individual points shown in the graph 601 indicate the determination index 216 about regions of interest included in the images 211 acquired at mutually different time points. The determination index 216 in the regions of interest included in the images 211 acquired from a time point t611 to a time point t612 and on or after a time point t613 is equal to or greater than the threshold 215. Thus, the biological-information calculating unit 206 calculates the biological information 212 from the pixel values of pixels included in the images 211 acquired from the time point t611 to the time point t612 and on or after the time point t613.

It is noted that a relatively lower threshold 215 possibly involves more inappropriate pixels. In contrast, a relatively higher threshold 215 is less likely to involve the determination index 216 equal to or greater than the threshold 215, thus possibly increasing the time required from the start of the imaging of the living organism 102 to the output of the biological information 212. Hence, the threshold 215 is preferably set in accordance with pixel quality allowable for the biological information 212, and latency time allowable by the living organism 102.

As described above, the measurement device 100 according to this preferred embodiment calculates the biological information 212 from time changes in a pixel value that is included in a region of interest included in the image 211 acquired by imaging the living organism 102, and that falls within a previously set range. The measurement device 100 according to this preferred embodiment can thus calculate the biological information 212 while preventing the accuracy of biological information calculation from lowering due to an adverse effect of an inappropriate pixel having a pixel value that is off from the previously set range.

### First Modification of First Preferred Embodiment

In a first modification of the measurement device 100 according to this preferred embodiment, the biological-information calculating unit 206 does not have to calculate the biological information 212 until the determination index 216 is kept equal to or greater than the threshold 215 during a predetermined time or a predetermined number of frames. Furthermore, the imaging unit 101 in this modification may image the living organism 102 until the determination index 216 becomes equal to or greater than the threshold 215. The biological-information calculating unit 206 in this case does not use the image 211 including a region of interest with the determination index 216 being lower than the threshold 215 in the process of calculating the biological information 212. The biological-information calculating unit 206 can thus calculate the biological information 212 from time changes in the pixel value of a pixel included in a region of interest with the determination index 216 being equal to or greater than the threshold 215. Biological information is reflected to the wave shape of a pulse wave; thus, using a temporally continuous wave shape in its entirety can calculate biological information more accurately than using a discontinuous wave shape.

For instance, let time changes in the determination index 216 be indicated by the graph 601, illustrated in FIG. 6. Furthermore, let the determination index 216 be kept equal to or greater than the threshold 215 during a predetermined number of frames on or after the time point t613. The biological-information calculating unit 206 in this modification in this case excludes the images 211 acquired from the time point t611 to the time point t612 from the process of calculating the biological information 212. The biological-information calculating unit 206 in contrast calculates the biological information 212 from the pixel values of pixels included in regions of interest included in the images 211 acquired at time points on or after the time point t613. The biological-information calculating unit 206 can consequently use the continuously acquired images 211 in the process of calculating the biological information 212. It is noted that whether to use the measurement device 100 according to this modification may be determined in accordance with latency time allowable by the living organism 102.

### Second Modification of First Preferred Embodiment

In a second modification of the measurement device 100 according to this preferred embodiment, the determination-index calculating unit 205 may calculate the determination index 216 by using the average, variance, standard deviation or other things of the pixel values of the target pixels TP included in a region of interest. For instance, the determination-index calculating unit 205 may calculate the determination index 216 on the basis of the average, variance, standard deviation or other things of the pixel values of the target pixels TP included in the region of interest, and on the basis of the number of target pixels TP included in the region of interest.

### Second Preferred Embodiment

A second preferred embodiment will be described. It is noted that identical or equivalent components in the drawings will be denoted by the same signs, and that the description of redundancies will be omitted. Configurations and processes having functions common substantially with those in the other preferred embodiments will be referred to with common symbols, and their description will be thus omitted; in addition, differences between this preferred embodiment and the other preferred embodiments will be described.

The predetermined lower limit 213 and predetermined upper limit 214 according to this preferred embodiment are set on the basis of at least one of an RGB color space and an HSV color space. To be specific, the predetermined lower limit 213 has a value based on at least one of the RGB color space and HSV color space. Likewise, the predetermined upper limit 214 has a value based on at least one of the RGB color space and HSV color space.

For instance, when the predetermined lower limit 213 is set on the basis of the RGB color space, a predetermined lower limit r1 for the pixel value of an R-subpixel, a predetermined lower limit g1 for the pixel value of a G-subpixel, and a predetermined lower limit b1 for the pixel value of a B-subpixel are set. That is, when the predetermined lower limit 213 is set on the basis of the RGB color space, the predetermined lower limit 213 is set for each of the R-, G- and B-subpixels.

When a region of interest includes an image of a spot, wrinkle, mole or other things of the living organism 102, an image of a blood vessel included in the region of interest is possibly mixed with the image of the spot, wrinkle, mole or other things of the living organism 102. The biological information 212 in this case cannot be possibly calculated appropriately from time changes in the pixel value of a pixel included in the region of interest. However, when a pixel value lower than the predetermined lower limit r1, predetermined lower limit g2 and predetermined lower limit b3 is a value indicating the color of the image of the spot, wrinkle, mole or other things of the living organism 102, the determination-index calculating unit 205 can exclude, from the target pixels TP, pixels included in the image of the spot, wrinkle, mole or other things of the living organism 102 included in the region of interest. The biological-information calculating unit 206 can consequently prevent the accuracy of calculation of the biological information 212 from degradation caused by an inappropriate pixel.

Alternatively, the predetermined lower limit 213, when set on the basis of the RGB color space, may be set using the pixel value of a subpixel of one color and the pixel value of a subpixel of another color. For |PixR(x,y)−PixG(x,y)|>m4 for instance, the determination-index calculating unit 205 determines a pixel including PixR(x,y), which is an R-subpixel, and PixG(x,y), which is a G-subpixel, as a target pixel TP. PixR(x,y) denotes the R-subpixel having x and y coordinates, PixG(x,y) denotes the G-subpixel having x and y coordinates, and m4 denotes the predetermined lower limit 213.

It is noted that the predetermined lower limit 213 and the predetermined upper limit 214 may be different between the R-, G- and B-subpixels.

Further, the pixel values of R, G and B indicated by the RGB color space are different greatly in some cases, depending on surrounding brightness at the time of imaging even when objects of the same color undergo imaging. In contrast to this, hue, saturation and value indicated by the HSV color space are less likely to be affected by the surrounding brightness at the time of imaging than the pixel values of R, G and B indicated by the RGB color space; thus, the determination-index calculating unit 205 may determine the target pixels TP on the basis of the hue, saturation and value indicated by the HSV color space when the surrounding brightness at the time of imaging is susceptible to fluctuations. For instance, the predetermined lower limit 213 and the predetermined upper limit 214 are set on the basis of the HSV color space, a predetermined lower limit h1 and a predetermined upper limit h2 both for the hue, a predetermined lower limit s1 and a predetermined upper limit s2 both for the saturation, and a predetermined lower limit b1 and a predetermined upper limit b2 both for the value are set.

The determination-index calculating unit 205 converts the pixel value of each pixel included in the region of interest from a value in the RGB color space into a value in the HSV color space when the predetermined lower limit 213 and the predetermined upper limit 214 are set on the basis of the HSV color space. The determination-index calculating unit 205 then determines whether the pixel value indicating the converted value in the HSV color space is equal to or greater than the predetermined lower limit 213 and equal to or smaller than the predetermined upper limit 214.

Determining the target pixels TP on the basis of the hue, saturation and value indicated by the HSV color space enables the determination-index calculating unit 205 to calculate the determination index 216 while preventing an effect caused by fluctuations in the surrounding brightness at the time of imaging.

Alternatively, the determination-index calculating unit 205 may determine the target pixels TP having a pixel value that is equal to or greater than the predetermined lower limit 213 corresponding to the RGB color space and is equal to or smaller than the predetermined upper limit 214 corresponding to the RGB color space, and that is equal to or greater than the predetermined lower limit 213 corresponding to the HSV color space and is equal to or smaller than the predetermined upper limit 214 corresponding to the HSV color space. It is noted that the predetermined lower limit 213 and predetermined upper limit 214 at this time are values corresponding to a color space; thus in some cases, a value corresponding to the RGB color space is different from a value corresponding to the HSV color space. The determination-index calculating unit 205 may then calculate the determination index 216 relating to the number of determined target pixels TP. The predetermined lower limit 213 and the predetermined upper limit 214 have their values based on the RGB color space and HSV color space, thus enabling the determination-index calculating unit 205 to select the target pixels TP having necessary information more accurately than it does in the case where the predetermined lower limit 213 and the predetermined upper limit 214 have their values based on a single color space.

It is noted that the color of a blood vessel image that is imaged by the imaging unit 101 is affected by the performance of a camera and the illumination level of the surrounding environment in some cases. Hence, the range of the pixel values of the target pixels TP based on at least one of the RGB color space and HSV color space may be set in accordance with the performance of the camera and the illumination level of the surrounding environment.

### Third Preferred Embodiment

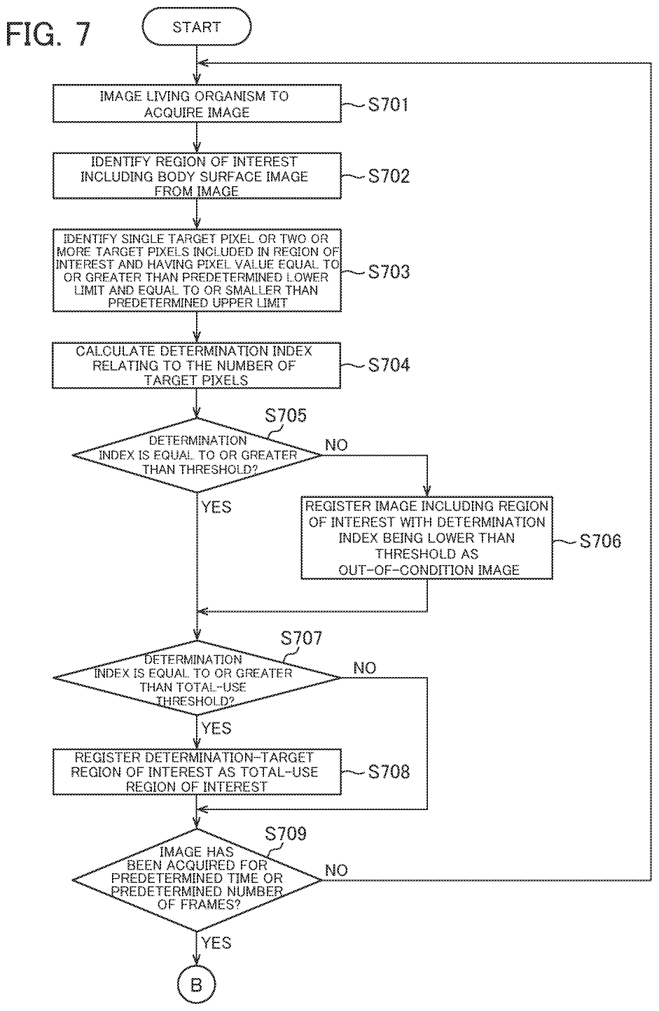

A third preferred embodiment will be described with reference to FIG. 7 to FIG. 9. It is noted that identical or equivalent components in the drawings will be denoted by the same signs, and that the description of redundancies will be omitted. Configurations and processes having functions common substantially with those in the other preferred embodiments will be referred to with common symbols, and their description will be thus omitted; in addition, differences between this preferred embodiment and the other preferred embodiments will be described.

The configuration of the measurement device 100 according to this preferred embodiment is illustrated in FIG. 1. The biological-information calculating unit 206 according to this preferred embodiment calculates the biological information 212 from the pixel values of all the pixels included in a region of interest with the determination index 216 being equal to or greater than a total-use threshold exceeding the threshold 215.

FIG. 7 is a flowchart illustrating an example operation of the measurement device 100 according to this preferred embodiment. The process steps in Steps S701 through S706 illustrated in FIG. 7, which are similar to those in Steps S301 through S306 illustrated in FIG. 3, will not be detailed.

In Step S707, the determination-index calculating unit 205 determines whether the determination index 216 is equal to or greater than the total-use threshold. If the determination index 216 is not equal to or greater than the total-use threshold in Step S707, the control unit 204 moves the process to Step S709. In contrast, if the determination index 216 is equal to or greater than the total-use threshold in Step S707, the determination-index calculating unit 205 in Step S708 registers the identification information of the image 211 including a determination-target region of interest into the storage unit 203 as the identification information of a total-use region of interest. The control unit 204 then moves the process to Step S709.

In Step S709, the biological-information calculating unit 206 determines whether the image 211 has been acquired for a predetermined time or a predetermined number of frames. The process step in Step S709, which is similar to that in Step S307 illustrated in FIG. 3, will not be detailed.

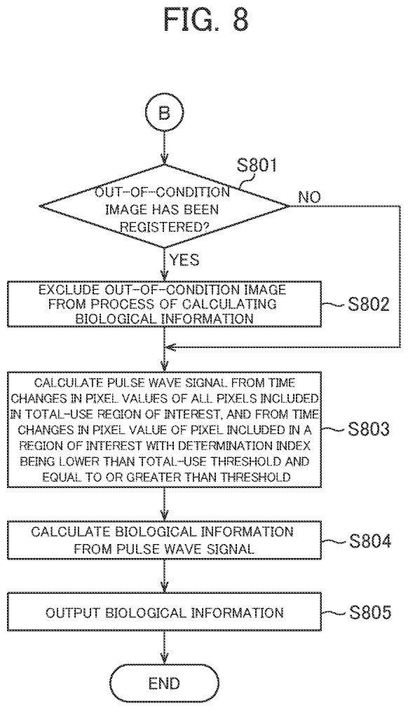

If the image 211 has not been acquired for the predetermined time or the predetermined number of frames in Step S709, the control unit 204 moves the process back to Step S701. In contrast, if the image 211 has been acquired for the predetermined time or the predetermined number of frames in Step S709, the control unit 204 moves the process to Step S801 illustrated in FIG. 8.

Next, the operation of the measurement device 100 according to this preferred embodiment will be still described with reference to FIG. 8. The process steps in Steps S801 through S802, which are similar to those in Steps S401 through S402 illustrated in FIG. 4, will not be detailed.

In Step S803, the biological-information calculating unit 206 calculates a pulse wave signal from time changes in the pixel values of all the pixels included in the total-use region of interest, and from time changes in the pixel values of the target pixels TP included in a region of interest with the determination index 216 being lower than the total-use threshold and equal to or greater than the threshold 215. The total-use region of interest is a region of interest included in the image 211 indicated by the identification information of the total-use region of interest registered in Step S708 illustrated in FIG. 7 and is a region of interest with the determination index 216 being equal to or greater than the total-use threshold. The control unit 204 then moves the process to Step S804. The process steps in Steps S804 through S805, which are similar to those in Steps S404 through S405 illustrated in FIG. 4, will not be detailed.

For instance, let the threshold 215 stand at 60%, and let the total-use threshold stand at 70%. The biological-information calculating unit 206 in this case uses, in the process of calculating the biological information 212, the pixel values of all the pixels included in a region of interest with the determination index 216 standing at 70% or greater. The biological-information calculating unit 206 also uses, in the process of calculating the biological information 212, the pixel values of the target pixels TP included in a region of interest with the determination index 216 standing at 60% or greater and less than 70%.

Although the numeric value 70% is non-limiting, it is preferable to set a numeric value such that a merit rather than an adverse effect that is exerted on measurement using a few inappropriate pixels is achieved greatly thanks to many pixels that are used. For instance, in using the average of the pixel values of pixels within a region of interest or other things to calculate biological information, using many appropriate pixels can achieve a signal quality improvement such as an improvement in signal noise ratio, thus improving the accuracy of biological information calculation in some cases even when data acquired by an image sensor constituting the imaging unit 101 includes inappropriate pixels.

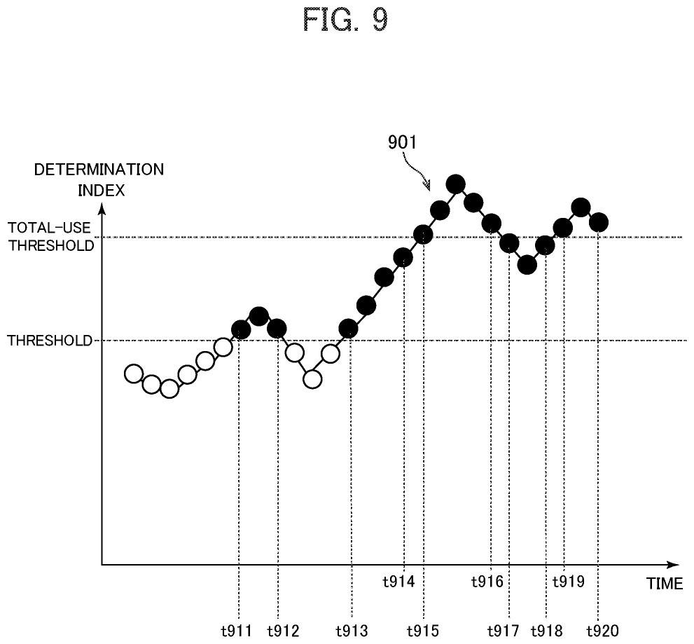

FIG. 9 illustrates a threshold, a total-use threshold, and a graph 901 showing example time changes in the determination index 216. The lateral axis in FIG. 9 indicates time, and the longitudinal axis in the same indicates the determination index. Individual points shown in the graph 901 indicate the determination index 216 about regions of interest included in the respective images 211. In response to the determination index 216 calculated as indicated by the graph 901, the biological-information calculating unit 206 uses, in the process of calculating the biological information 212, time changes in the pixel values of all the pixels in a region of interest included in each of the images 211 acquired at a time point t915 to a time point t916 and a time point t919 to a time point t920. For instance, the biological-information calculating unit 206 uses, in the process of calculating a pulse wave signal, time changes in the pixel values of all the pixels in the region of interest included in each of the images 211 acquired at the time point t915 to the time point t916 and the time point t919 to the time point t920.

Furthermore, in response to the determination index 216 calculated as illustrated in the graph 901, the biological-information calculating unit 206 uses, in the process of calculating the biological information 212, time changes in the pixel values of the target pixels TP included in regions of interest of the images 211 acquired at a time point t911 to a time point t912, a time point t913 to a time point t914 and a time point t917 to a time point t918. For instance, the biological-information calculating unit 206 uses, in the process of calculating the pulse wave signal, time changes in the pixel values of the target pixels TP included in the regions of interest of the images 211 acquired at the time point t911 to the time point t912, the time point t913 to the time point t914 and the time point t917 to the time point t918.

As such, the biological-information calculating unit 206 uses, in the process of calculating the biological information 212, the pixel values of all the pixels included in a region of interest with the determination index 216 being equal to or greater than the total-use threshold. There are at this time fewer inappropriate pixels than appropriate pixels to such a degree that the determination index 216 satisfies the total-use threshold, thus predominating improvement in calculation accuracy resulting from use of many appropriate pixels for calculation over accuracy degradation resulting from use of inappropriate pixels. The measurement device 100 according to this preferred embodiment can consequently calculate the biological information 212 by using the pixel values of many appropriate pixels and can thus improve the accuracy of calculation of the biological information 212.

### Fourth Preferred Embodiment

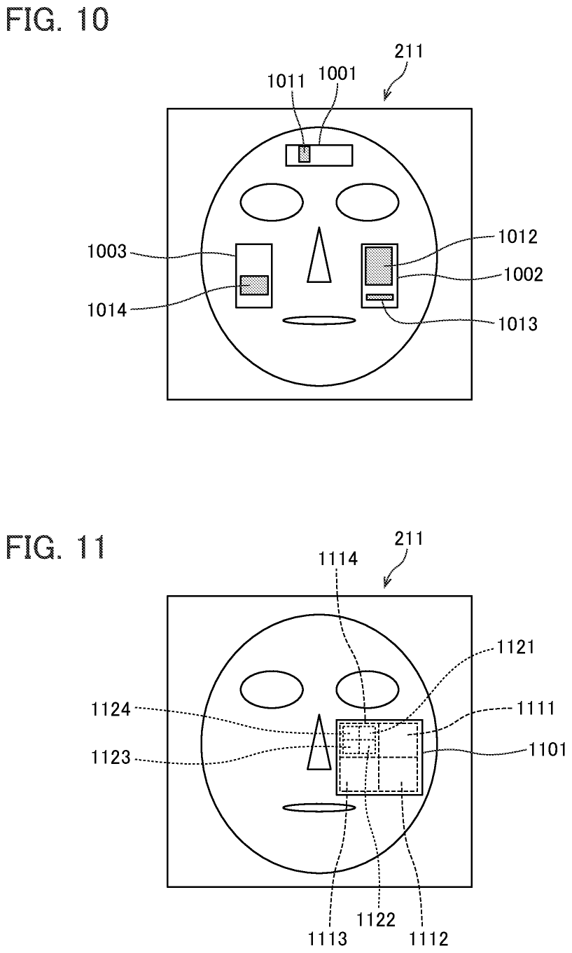

A fourth preferred embodiment will be described with reference to FIG. 10. It is noted that identical or equivalent components in the drawings will be denoted by the same signs, and that the description of redundancies will be omitted. Configurations and processes having functions common substantially with those in the other preferred embodiments will be referred to with common symbols, and their description will be thus omitted; in addition, differences between this preferred embodiment and the other preferred embodiments will be described.

The configuration of the measurement device 100 according to this preferred embodiment is illustrated in FIG. 1.

The determination-index calculating unit 205 according to this preferred embodiment calculates the determination index 216 for each of a plurality of regions of interest included in the image 211.

The biological-information calculating unit 206 according to this preferred embodiment calculates the biological information 212 from the pixel value of a pixel included in a region of interest with the determination index 216 being equal to or greater than the threshold 215 among the plurality of regions of interest included in the image 211. To be specific, the biological-information calculating unit 206 calculates the biological information 212 from the pixel values of the target pixels TP included in a region of interest with the determination index 216 being equal to or greater than the threshold 215 among the plurality of regions of interest included in the image 211.

Alternatively, the biological-information calculating unit 206 may calculate the biological information 212 from the pixel values of the target pixels TP included in a region of interest with the determination index 216 being the largest of the plurality of regions of interest included in the image 211.

Alternatively, the biological-information calculating unit 206 may calculate the biological information 212 from the pixel values of all the pixels included in a region of interest with the determination index 216 being equal to or greater than a total-use threshold among the plurality of regions of interest included in the image 211.

FIG. 10 illustrates a plurality of example regions of interest included in the image 211. Let pixels constituting a region 1011 included in a region of interest 1001, pixels constituting a region 1012 included in a region of interest 1002, pixels constituting a region 1013 included in the region of interest 1002, and pixels constituting a region 1014 included in a region of interest 1003 be defined as non-target pixels NTP. Moreover, let the determination index 216 in the region of interest 1001 and the determination index 216 in the region of interest 1003 be equal to or greater than the threshold 215. Further, let the determination index 216 in the region of interest 1002 be lower than the threshold 215. The biological-information calculating unit 206 in this case calculates the biological information 212 from the pixel values of the pixels included in the region of interest 1001 and the region of interest 1003. The biological-information calculating unit 206 herein excludes the pixels constituting the region 1011 and the pixels constituting the region 1014 from the process of calculating the biological information 212 and then calculates the biological information 212. It is noted that in an image 211 different from the image 211 illustrated in FIG. 10, the biological-information calculating unit 206 excludes this different image 211 from the process of calculating the biological information 212 when the determination index 216 is lower than the threshold 215 in any of the regions of interest 1001 to 1003.

As described above, the measurement device 100 according to this preferred embodiment can calculate the biological information 212 from the pixel values of the target pixels TP included in a region of interest suitable for calculating the biological information 212 among the plurality of regions of interest.

### Modification of Fourth Preferred Embodiment

In a modification of the measurement device 100 according to this preferred embodiment, a region of interest may include a plurality of divided regions of interest.

FIG. 11 illustrates a plurality of example regions of interest included in a region of interest. A region of interest 1101 illustrated in FIG. 11 includes a region of interest 1111 to a region of interest 1114. The region of interest 1111 to the region of interest 1114 are regions with the region of interest 1101 divided. Furthermore, the region of interest 1114 includes a region of interest 1121 to a region of interest 1124. The region of interest 1121 to the region of interest 1124 are regions with the region of interest 1114 divided.

When the determination index 216 is lower than the threshold 215 in a region of interest that is a determination target, the determination-index calculating unit 205 according to this modification calculates the determination index 216 for a plurality of regions of interest composed of this determination-target region of interest divided. For instance, when the determination index 216 is lower than the threshold 215 in the region of interest 1101, the determination-index calculating unit 205 calculates the determination index 216 for each of the regions of interest 1111 to 1114.

Furthermore for instance, when the determination index 216 is lower than the threshold 215 in the regions of interest 1111 to 1114, the determination-index calculating unit 205 calculates the determination index 216 for each of a plurality of regions of interest with the regions of interest 1111 to 1114 divided. For instance, the determination-index calculating unit 205 calculates the determination index 216 for each of the regions of interest 1121 to 1124. As described above, the determination-index calculating unit 205 calculates the determination index 216 for a divided determination-target region of interest until the determination index 216 becomes equal to or greater than the threshold 215 in the determination-target region of interest having a predetermined size or larger size.

The biological-information calculating unit 206 then calculates the biological information 212 from the pixel value of a pixels included in the divided determination-target region of interest when the calculated determination index 216 is equal to or greater than the threshold 215 in the divided determination-target region of interest. That is, the biological-information calculating unit 206 calculates the biological information 212 from the pixel value of a pixel included in a region of interest having a predetermined minimum size or larger size and with the determination index 216 being equal to or greater than the threshold 215. For instance, when the region of interest 1124 has a predetermined size or larger size, and when the determination index 216 in the region of interest 1124 is equal to or greater than the threshold 215, the biological-information calculating unit 206 calculates the biological information 212 from the pixel value of a pixel included in the region of interest 1124.

As such, even when the determination index 216 in a relatively large region of interest is lower than the threshold 215, the biological-information calculating unit 206 according to this modification calculates the biological information 212 from the pixel value of a pixel included in a region of interest included in the relatively large region of interest and with the determination index 216 being equal to or greater than the threshold 215.

As described above, the measurement device 100 according to this modification can calculate the biological information 212 from the pixel value of a target pixel included in a region of interest where the biological information 212 can be calculated accurately within a relatively large region of interest even when the determination index 216 in the relatively large region of interest is lower than the threshold 215.

### Fifth Preferred Embodiment

A fifth preferred embodiment will be described. It is noted that identical or equivalent components in the drawings will be denoted by the same signs, and that the description of redundancies will be omitted. Configurations and processes having functions common substantially with those in the other preferred embodiments will be referred to with common symbols, and their description will be thus omitted; in addition, differences between this preferred embodiment and the other preferred embodiments will be described.

In the measurement device 100 according to this preferred embodiment, at least any selected from the group consisting of the predetermined lower limit 213, predetermined upper limit 214 and threshold 215 has a value corresponding to the type of the biological information 212 that is a calculation target.

The predetermined lower limit 213, predetermined upper limit 214 and threshold 215 associated with the type of this calculation-target biological information 212 are stored in the storage unit 203 according to this preferred embodiment.

For instance, for pulse calculation in the biological-information calculating unit 206, the reliability of the calculated biological information 212 tends to be able to be satisfied even when pixels within a region of interest include relatively many pixels containing unnecessary information other than blood vessels when compared with an instance of blood pressure calculation. That is, when the type of the biological information 212 is pulse, there is a tendency where the difference between the predetermined upper limit 214 and predetermined lower limit 213 may be relatively large when compared with an instance where the type of the biological information 212 is blood pressure. Likewise, when the type of the biological information 212 is pulse, there is a tendency where the threshold 215 may be relatively low when compared with the instance where the type of the biological information 212 is blood pressure. That is, the range of a pixel value that is required, and the determination index 216 that is needed vary in accordance with the type of biological information that is a calculation target.

However, the predetermined lower limit 213 and predetermined upper limit 214 varying from type to type of the biological information 212 that is a calculation target are stored in the storage unit 203, thereby enabling the determination index 216 to be calculated based on a criterion corresponding to the type of the biological information 212. Furthermore, the threshold 215 varying from type to type of the biological information 212 is stored in the storage unit 203, thereby enabling the biological-information calculating unit 206 to determine, on the basis of the threshold 215 corresponding to the type of the biological information 212, a region of interest and the image 211 that are used for calculating the biological information 212. The measurement device 100 can consequently calculate the biological information 212 from the pixel value of a pixel included in a region of interest, on the basis of a criterion corresponding to the type of the biological information 212.

### Sixth Preferred Embodiment

A sixth preferred embodiment will be described. It is noted that identical or equivalent components in the drawings will be denoted by the same signs, and that the description of redundancies will be omitted. Configurations and processes having functions common substantially with those in the other preferred embodiments will be referred to with common symbols, and their description will be thus omitted; in addition, differences between this preferred embodiment and the other preferred embodiments will be described.

In the measurement device 100 according to this preferred embodiment, at least one selected from the group consisting of the predetermined lower limit 213 and predetermined upper limit 214 has a value corresponding to a body surface color associated with a type to which the living organism 102 belongs. Examples of the type to which the living organism 102 belongs include the age and race of the living organism 102.

For instance, the color, saturation, value and other things of an image of an imaged blood vessel tend to vary in accordance with age, such as childhood, adolescence, the age of maturity and old age. However, the predetermined lower limit 213 and predetermined upper limit 214 corresponding to the age are associated and stored in the storage unit 203, thereby enabling the determination-index calculating unit 205 to calculate the determination index 216 appropriately in accordance with the age of the living organism 102.

As described above, the measurement device 100 can determine the target pixels TP on the basis of the type to which the living organism 102 belongs by storing the predetermined lower limit 213 and predetermined upper limit 214, varying from type to type to which the living organism 102 belongs, in the storage unit 203. The measurement device 100 according to this preferred embodiment can consequently calculate the biological information 212 from the pixel values of the target pixels TP corresponding to the age, race and other things of the living organism 102.

### Modification of Sixth Preferred Embodiment

In a modification of the measurement device 100 according to this preferred embodiment, at least one selected from the group consisting of the predetermined lower limit 213 and predetermined upper limit 214 may have a value corresponding to a body surface color associated with the type to which the living organism 102 belongs, and the threshold 215 may have a value corresponding to the type of the biological information 212 that is a calculation target. The measurement device 100 according to this modification in this case can determine the target pixels TP on the basis of a criterion corresponding to the type to which the living organism 102 belongs and can calculate the determination index 216 on the basis of a criterion corresponding to the type of this calculation-target biological information 212. The measurement device 100 according to this modification can consequently calculate the biological information 212 from the pixel value of a pixel included in a region of interest, on the basis of a criterion corresponding to the type to which the living organism 102 belongs, and corresponding to the type of the calculation-target biological information 212.

The individual processes that are executed in the foregoing preferred embodiments are not limited to the process aspects illustrated in the respective preferred embodiments. The foregoing function blocks may be implemented with either a logic circuit (hardware) formed in, but not limited to, an integrated circuit or software using a CPU. The individual processes that are executed in the foregoing preferred embodiments may be executed by a plurality of computers. For instance, some of the processes that are executed in the respective function blocks of the control unit 204 of the measurement device 100 may be executed by another computer, or all of the processes may be shared among the plurality of computers.

The present disclosure is not limited to the foregoing individual preferred embodiments. Various modifications can be devised with the scope of the claims. A preferred embodiment that is obtained in combination, as appropriate, with the technical means disclosed in the respective preferred embodiments is also included in the technical scope of the present disclosure. Furthermore, combining the technical means disclosed in the respective preferred embodiments can form a new technical feature.

What is claimed is:

1. A measurement device comprising:
an imaging unit configured to image a living organism to acquire an image;
a determination-index calculating unit configured to calculate, for a region of interest included in the image, a determination index relating to a pixel count of a target pixel having a pixel value equal to or greater than a predetermined lower limit and equal to or smaller than a predetermined upper limit; and
a biological-information calculating unit configured to calculate biological information from a pixel value of a pixel included in a region of interest with the determination index being equal to or greater than a threshold.

2. The measurement device according to claim 1, wherein
the predetermined lower limit has a value corresponding to a lower limit of a dynamic range of the image, and
the predetermined upper limit has a value corresponding to an upper limit of the dynamic range.

3. The measurement device according to claim 1, wherein the biological-information calculating unit determines an out-of-condition image including a region of interest with the determination index being lower than the threshold, or a moving image including the out-of-condition image as an imaging failure.

4. The measurement device according to claim 3, wherein the biological-information calculating unit excludes the out-of-condition image, or the moving image including the out-of-condition image from a process of calculating the biological information.

5. The measurement device according to claim 1, wherein the biological-information calculating unit calculates the biological information from pixel values of all pixels included in a region of interest with the determination index being equal to or greater than a total-use threshold exceeding the threshold.

6. The measurement device according to claim 1, wherein the biological-information calculating unit calculates the biological information from the pixel value of the target pixel included in a region of interest with the determination index being equal to or greater than the threshold.

7. The measurement device according to claim 1, wherein
the determination-index calculating unit calculates the determination index for each of a plurality of regions of interest included in the image, and
the biological-information calculating unit calculates the biological information from a pixel value of a pixel included in a region of interest with the determination index being equal to or greater than the threshold among the plurality of regions of interest.

8. The measurement device according to claim 1, wherein
the predetermined lower limit has a value based on at least one of an RGB color space and an HSV color space, and
the predetermined upper limit has a value based on at least one of the RGB color space and the HSV color space.

9. The measurement device according to claim 1, wherein the biological-information calculating unit does not calculate the biological information until the determination index is kept equal to or greater than the threshold during a predetermined time or a predetermined number of frames.

10. The measurement device according to claim 1, wherein the imaging unit images the living organism until the determination index becomes equal to or greater than the threshold.

11. The measurement device according to claim 1, wherein at least any selected from the group consisting of the predetermined lower limit, the predetermined upper limit and the threshold has a value corresponding to a type of the biological information.

12. The measurement device according to claim 1, wherein at least one selected from the group consisting of the predetermined lower limit and the predetermined upper limit has a value corresponding to a body surface color associated with a type to which the living organism belongs.

13. A measurement method comprising the steps of:
imaging a living organism to acquire an image;
calculating, for a region of interest included in the image, a determination index relating to a pixel count of a target pixel having a pixel value equal to or greater than a predetermined lower limit and equal to or smaller than a predetermined upper limit; and
calculating biological information from a pixel value of a pixel included in a region of interest with the determination index being equal to or greater than a threshold.

14. A computer-readable recording medium recording a program that is executed by a computer, the program comprising the functions of:
imaging a living organism to acquire an image;
calculating, for a region of interest included in the image, a determination index relating to a pixel count of a target pixel having a pixel value equal to or greater than a predetermined lower limit and equal to or smaller than a predetermined upper limit; and
calculating biological information from a pixel value of a pixel included in a region of interest with the determination index being equal to or greater than a threshold.

15. The measurement device according to claim 2, wherein the biological-information calculating unit determines an out-of-condition image including a region of interest with the determination index being lower than the threshold, or a moving image including the out-of-condition image as an imaging failure.

16. The measurement device according to claim 15, wherein the biological-information calculating unit excludes the out-of-condition image, or the moving image including the out-of-condition image from a process of calculating the biological information.
